(19) United States
(12) Patent Application Publication
(10) Pub. No.: US 2021/0177339 A1
(43) Pub. Date: Jun. 17, 2021

(54) SYSTEM AND METHOD FOR EARLY DETECTION OF SEPSIS

(71) Applicant: DIGNITY HEALTH, Phoenix, AZ (US)

(72) Inventors: Joseph Colorafi, Phoenix, AZ (US); Alyson D'Andrea, Phoenix, AZ (US); Sunil Kumar Kakade, Phoenix, AZ (US); Umesh Kalyanasundaram, Phoenix, AZ (US); Murali Nandula, Phoenix, AZ (US); Gaurav Padiyar, Phoenix, AZ (US); Ken Ferrell, Phoenix, AZ (US); Monica Spoerer, Phoenix, AZ (US); Harry Schned, Phoenix, AZ (US)

(21) Appl. No.: 17/118,944

(22) Filed: Dec. 11, 2020

Related U.S. Application Data
(60) Provisional application No. 62/948,048, filed on Dec. 13, 2019.

(51) Int. Cl. A61B 5/00

(52) U.S. Cl. CPC A61B 5/412; A61B 5/746; A61B 5/742; A61B 5/0022; A61B 5/0004; A61B 5/747

(57) ABSTRACT

The disclosed embodiments include a sepsis alert and recovery treatment system that is configured to provide identification of time zero, track documentation and communication between providers identifying sepsis, generate associated alerts, provide recommendations and tracking for SEP-1 bundle compliance, display remaining time for reassessment, and recommend and track re-assessment and sepsis recovery.

### CROSS REFERENCE TO RELATED APPLICATIONS

This application claims the benefit of U.S. Provisional Patent Application Ser. No. 62/948,048, filed Dec. 13, 2019, the disclosure of which is incorporated herein by reference in its entirety for all purposes.

### BACKGROUND

Sepsis is a multifaceted complication of infection that is life threatening. Sepsis is typically conceptualized as a three phase syndrome that progresses to a diagnosis of severe sepsis, septic shock, and/or systemic inflammatory response syndrome (SIRS), These diagnoses may result in organ failure, and sometimes death. The diagnosis of severe sepsis is difficult given its multiple diagnostic criteria, which generally include altered mental status, abnormal readings or levels for vital signs, and organ dysfunction with a probable or confirmed infection.

Each year in the United States more than 1.5 million people acquire sepsis and 250,000 die from sepsis. One in three people who die in a U.S. hospital have sepsis. Thus, given its prevalence, clinicians, doctors, nurses, or other healthcare professionals or providers providing inpatient care, referred to herein as providers, are especially vigilant in monitoring patients for sepsis.

The Surviving Sepsis Campaign was started in 2002 with the goal of reducing mortality from sepsis and improving early diagnosis. This organization was instrumental in the review and distribution of evidence-based guidelines (“sepsis bundles”) for the care of patients with sepsis, many of which are configured into in-patient electronic medical records (EMR) in the form of clinical decision support alerts and electronic order sets. The reporting of timely implementation of evidence based treatment guidelines, known as the SEP-1 bundle, is a non-limiting example of one such reportable process of care measure and involves the calculation of a starting point known as time zero.

Time zero may be defined as the time of presentation of the conditions of sepsis. Time zero may reflect the time of triage in the emergency department for someone who presents with sepsis or from documentation in the chart for someone who develops symptoms of sepsis while hospitalized. Assigned retrospectively, time zero allows the abstractor to calculate the time when sepsis was first identified to when it was diagnosed and therapy was initiated. Time zero is an important part of sepsis diagnosis, since, according to the guidelines discussed above, providers have 180 minutes from the identification of time zero to begin certain treatments of the identified sepsis.

In light of the above, and the fact that one in three people who die in hospitals have sepsis, real-time automated surveillance for sepsis among hospitalized patients is of critical importance. The early identification and treatment of sepsis is associated with reduced mortality and costly intensive care bed days.

Regarding mortality, in situations where a patient has contracted sepsis (or in emergency situations generally) time is of the essence. An early detection of sepsis prevents negative impacts on the patient such as the loss of vital organs and/or the associated fatalities from sepsis. Thus, the sooner medical professionals are able to detect and monitor sepsis, the better for the patient.

Regarding cost, the onset and treatment of sepsis is one of the highest expenses for hospitals in the United States, each case costing approximately $10,000. Furthermore, the longer patients stay in the hospital, the greater the treatment cost involved. The medical industry is therefore actively trying to, and has to some degree had success in gaining control of sepsis cases, in reducing the mortality by almost half, and in reducing the associated cost.

With this in mind, a rapid delivery of critical information is important. In most situations, the critical data is stored within an EMR, but the EMR does not include features giving providers all consolidated information in one place, or the associated levels for all patients. In order to perform a proper diagnosis, monitoring, and treatment of a patient, health care professionals must manually access various medical panels (e.g., lactate panels), previous orders of tests, etc. The providers must then manually calculate the onset of sepsis, to determine at what point the sepsis began affecting the patient. Once the onset of sepsis has been determined, the providers must then formulate a strategy for treating each patient. The use of time to access medical records and make the relevant calculations therefore puts the focus on administrative tasks and manual analysis of existing data, and draws attention away from the patient.

Thus, what is needed is a system that provides for 1) early detection of sepsis, 2) reducing the cost and time of patients who have contracted sepsis staying in the ICU, 3) improving monitoring of conditions by medical staff, and 4) increasing the recovery speed for patients who no longer have sepsis and have left the ICU, sometimes referred to as sepsis recovery.

No solutions in the current state of the art include a collection of real time monitored data that demonstrates all available sepsis data within the data repository simultaneously. Thus, no solutions include an automated sepsis time zero or a dashboard that quickly summarizes data from a variety of sources to determine a strategy for compliance with bundles such as a SEP-1 bundle.

### SUMMARY

The present disclosure overcomes the aforementioned drawbacks by providing a multi-faceted program to improve the early identification of sepsis and initiate timely, evidence-based care to treat sepsis, two events that are associated with decreased sepsis mortality.

The ultimate goal of the disclosed invention is to reduce mortality due to severe sepsis across the organization (i.e., as a collaborative team effort) by identifying possible signs and symptoms of sepsis early and treating the patient in a timely manner. Then, to support the patient through the recovery phase to transfer or discharge to reduce overall length of stay.

Specifically, the disclosed embodiments include a sepsis alert and recovery treatment system that is configured to provide identification of time zero, track documentation and communication identifying sepsis from nurses or other providers to doctors, generate associated alerts, provide recommendations and tracking for SEP-1 bundle compliance, display remaining time for reassessment, and recommend and track re-assessment and sepsis recovery.

To address the needs of each patient in need of the sepsis bundles described above, the disclosed embodiments include a web or other server/client application which in real time compiles the data from EMR or ADT systems and displays, possibly on large screens, the compiled information in emergency or ICU departments, allowing multiple providers to view screens within hospitals, so that the information is constantly available to them, and they are able to make faster decisions and administer to the patients immediately rather than accessing the EMR system, selecting a patient, selecting different menus, observing current treatment, and so forth.

The automated algorithms of the disclosed embodiments may be used to improve several key indicators including superior positive predictive value, enhanced timing and performance of a predictive sepsis alert for providers provided through a system developed for desktop or mobile applications, within a critical 180-minute SEP-1 window, increase bundle compliance, reduce ICU length of stay, and ultimately decrease mortality from sepsis. The disclosed embodiments further provide an improvement to performance of EMR and/or ADT access and algorithms to provide early indications of sepsis and early intervention to improve evidence based bundle compliance, in response to the real-time, automated time zero calculation.

### BRIEF DESCRIPTION OF THE DRAWINGS

FIG. 1 is a block diagram of a system configured to implement the present disclosure.

FIG. 2 is a flow chart setting forth the steps of processes for diagnosing a patient for sepsis, calculating time zero, providing bundle compliance recommendations, generating an alert, monitoring communications and monitoring of the patient, tracking recovery time zero and compliance, and displaying the above on a user interface in near real time, in accordance with the present disclosure.

FIG. 3 is a more detailed flow chart setting forth the steps of calculating time zero, in accordance with the present disclosure.

FIG. 4 is a more detailed flow chart setting forth the steps of calculating recovery time zero, in accordance with the present disclosure.

FIG. 5 is a non-limiting example interface used in displaying and monitoring bundle compliance, in accordance with the present disclosure.

FIG. 6 is a non-limiting example interface used in displaying and monitoring bundle compliance, in accordance with the present disclosure.

FIG. 7 is a non-limiting example interface used in displaying and monitoring bundle compliance, in accordance with the present disclosure.

FIG. 8 is a non-limiting example interface used in displaying and monitoring bundle compliance, in accordance with the present disclosure.

FIG. 9 is a non-limiting example interface used in displaying and monitoring bundle compliance, in accordance with the present disclosure.

FIG. 10 is a non-limiting example interface used in displaying and monitoring bundle compliance, in accordance with the present disclosure.

FIG. 11 is a non-limiting example interface used in displaying and monitoring recovery bundle compliance, in accordance with the present disclosure.

FIG. 12 is a non-limiting example interfaces used in displaying and monitoring recovery bundle compliance, in accordance with the present disclosure.

FIG. 13 is a non-limiting example interfaces used in displaying retrospective data, in accordance with the present disclosure.

FIG. 14 is a non-limiting example interfaces used in displaying retrospective data, in accordance with the present disclosure.

FIG. 15 is a non-limiting example interfaces used in displaying retrospective data, in accordance with the present disclosure.

FIG. 16 is a non-limiting example interfaces used in displaying retrospective data, in accordance with the present disclosure.

### DETAILED DESCRIPTION

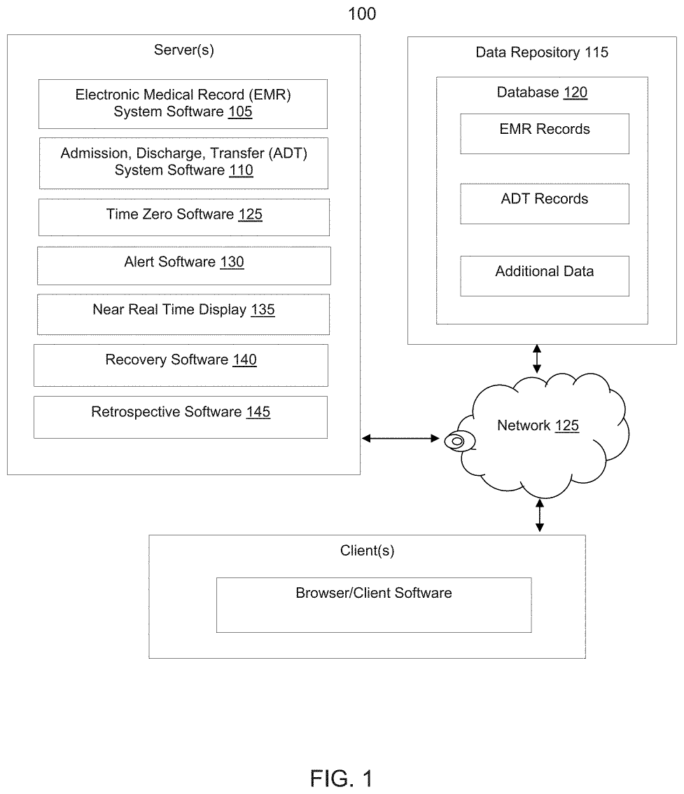

The disclosed system may be configured to execute several algorithms, which drive the system to collect data, store the data within a data platform, and use that data to make decisions regarding the treatment of sepsis patients, then transition them into recovery. Specifically, referring particularly now to FIG. 1, a streamlined block diagram of a sepsis alert and recovery treatment system 100 is shown that is configured to provide identification of time zero, track documentation and communication regarding the indication of sepsis details between providers, generate associated alerts, display remaining time for reassessment, provide recommendations and tracking for SEP-1 bundle compliance, and recommend and monitor re-assessment and sepsis recovery.

In general, as shown in FIG. 1, the sepsis alert and recovery treatment system 100 includes one or more servers running one or more Electronic Medical Record (EMR) software modules 105, one or more Admission, Discharge, Transfer (ADT) software modules 110, and a data repository 115, possibly including one or more databases 120. Additional software modules within the disclosed system may include: one or more time zero calculation software engines or modules 125; one or more alert software modules 130; one or more near real time display software modules 135; one or more recovery software modules 140; and one or more retrospective software modules 145.

The components of the sepsis alert and recovery treatment system 100 may be located on the same device, as shown in FIG. 1, including, but not limited to, a server, mainframe, desktop Personal Computer (PC), laptop, Personal Digital Assistant (PDA), telephone, mobile phone, kiosk, cable box, and the like. Alternatively, the components of the sepsis alert and recovery treatment system 100 may be located on separate devices connected by a network 125 (e.g., the Internet), with wired and/or wireless segments.

For purposes of this disclosure, the terms server and/or server computer may refer to any combination of the components of the sepsis alert and recovery treatment system 100, running any of the algorithms for the software modules and/or software engines described herein. For example, servers may include one or more software modules or software engines, and their associated algorithms, executing on one or more hardware computing devices, such as a server computer aggregating the data in the data repository 115, identifying time zero, creating and populating a SEP-1 template for compliance, identify recovery time zero, making these determinations available through interfaces, such as using an API, or rendering and transmitting a user interface on a web page using a server-side graphical user interface module such as the near real time display 135, and displaying the interface on a client computer using a client-side graphical user interface module, as non-limiting examples.

The server computer(s) may therefore execute the method steps within one or more of the disclosed algorithms by sending instructions, possibly in the form of compiled and executable software code for any of the disclosed software modules, to a processor on the server computer(s). The processor may then execute these instructions causing the server computer(s) to complete the disclosed method steps.

The data repository 115 may be configured to store data associated with the sepsis alert and recovery treatment system 100. The data repository 115 may be any sort of system capable of storing data, such as a relational database, a database management system, a hierarchical file, a flat file, and the like. The data repository 115 may be directly connected with any of the disclosed software modules, and/or to the server computers themselves, through various network configurations (e.g., wired, wireless, LAN, WAN, etc.). In one non-limiting example, the data may further include healthcare data associated with patients admitted to healthcare facilities.

The data repository 115 seen in FIG. 1 may be made up of a big data platform, sometimes referred to as a “data lake.” Data from any available enterprise-wide health information systems may be aggregated into this data repository 115. As a non-limiting example, the data platform may include Cloudera, implementing SAS and/or an instance of Hadoop. In this non-limiting example, Hadoop may provide open source, flat-file software that allows for the distributed processing of large datasets. Likewise, SAS may provide, for example Visual Analytics tools to aid in data exploration and display.

The disclosed system may further include an EMR system 105. The EMR system may comprise any combination of data and software logic used to capture any personal, documentation, clinical, and/or treatment data acquired as patients are admitted to, diagnosed, and treated within, a hospital or other healthcare facility. Typically, in order to access and analyze patient data, providers must access the EMR system 105. While this allows providers access to data information regarding patients, if the providers need this data in real time from the EMR system 105, they may access it as it is entered into the EMR system 105, but must do so manually.

The disclosed system may further include an Admissions, Discharge, and Transfer (ADT) system 110. The ADT system may be configured to track multiple types of data, including the reasons that patients were admitted, as well as admission diagnosis information, access to data from nursing notes, patient vitals, etc. The ADT system may then store the collected data in standard discreet values. The ADT system 110 also includes records noting when and why patients were discharged, and/or transferred. Like the EMR system 105 above, typically, in order to access and analyze patient data, providers must access the ADT system 110. While this allows providers access to data information regarding patients, if the providers need this data in real time from the ADT system 110, they may access it as it is entered into the ADT system 110, but must do so manually.

By contrast, the disclosed embodiments include a near real time display system 135, allowing providers to instantly have access to the data within the EMR system 105, ADT system 110, and any additional data stored in the data repository 115.

No limitations should be placed on the type of interface to be used by the disclosed embodiments. As non-limiting examples, interfaces may include: a graphical user interface (GUI) that is displayed by a browser or other client-side software; an API model, in which an API receives one or more Remote Procedure Calls (RPC) from a user and/or additional software, and generates response data to be used in any possible data format (e.g., data elements populated into another app or web technology), or into data repository 115; or populating a Computer Telephony Integration (CTI) within a call center, as non-limiting examples.

In one non-limiting example, the sepsis alert and recovery treatment system 100 may include multiple interfaces that may be accessed and manipulated by multiple users simultaneously, such as an API model, in which users, other devices, and/or software performing RPCs access the one or more available interfaces. Similarly, various instances of the interface may be accessed on multiple browsers or using an RPC from additional software, possibly client-side software. Additionally, the sepsis alert and recovery treatment system 100 may be optionally connected to data repository 115 and/or multiple databases 120. The sepsis alert and recovery treatment system 100 may connect to the data repository 115 and/or databases 120 physically and/or over a network 150, for example.

In embodiments that include displaying a sepsis dashboard/user interface on a browser on a client machine, the browser may be configured to display the dashboard on a user interface, which may be a graphical user interface (GUI) associated with the sepsis alert and recovery treatment system 100. Those skilled in the art, having benefit of this detailed description, will appreciate that there will be many ways in which a GUI may be viewed other than using a browser.

To provide a centralized data repository providing all accessible data, the disclosed system may provide for software that receives the data via the EMR system 105 or ADT system 110, or any other enterprise-wide health information systems, and drives or pushes the data from the multiple source systems to the data repository 115. As the data is updated, the data may also be updated in real time in the data repository 115.

As non-limiting examples, the data received and aggregated within the data repository 115 may include provider documentation, clinical data, surgical history, and medication data (e.g., medications given). Additional general data may include notes or other user input demonstrating the reason for the patient's visit to and/or admission to the hospital or healthcare facility, and any additional insights that the provider may have.

As non-limiting examples, provider documentation, possibly gathered when the patient is admitted, may include: general data; patient personal data (e.g., patient's name, age, ethnicity, address, phone number, insurance, emergency contacts, etc.); social history; smoking history; notes from the admitting provider; a patient's reason for visiting the healthcare facility, or associated problems; doctors' or nurses' notes regarding the patient's visit; nursing or other provider general documentation; notes regarding a patient's mental status; a doctor or other provider's diagnosis; provider's observations; medical history; placement of an order by the doctor or other provider, and the like.

As non-limiting examples, clinical data may include initial or ongoing examinations and/or observations, such as: vital signs determined by a doctor or on-site equipment generally; patient's temperature; patient heart rate; patient blood pressure; patient pulse ox (pO2); patient respiratory rate; patient glucose level; patient white blood cell count; patient platelet count; patient bilirubin count; patient lactate level; patient creatinine level; patient INR level; patient Partial Thromboplastin Time (PTT)/Prothrombin Time (PT, indicating abnormal times for blood to clot); patient renal and hepatic function (e.g., whether renal or hepatic function is impaired); patient POSS score; patient RASS score; patient MOSS score; providers' diagnoses; and the like.

Non-limiting examples of patient surgical history may include: patient surgery case; patient surgery case procedure; Abdominal or Thoracic surgery performed on the patient; patient anesthesia time (e.g., has anesthesia been administered greater than 3 hours previously, or in the last 24 hours); and the like.

As non-limiting examples medication data may include: medications given; opioid or benzo medication administration; diagnoses that prompted the medication being prescribed, medication documentation; whether a sedative was given less than 2 hours ago; whether anesthesia time was greater than 3 hours or within the last 24 hours; and the like.

It should be noted that although the non-limiting examples above are grouped, these groups are for example purposes only. Any of the data above may be included in any group. Furthermore, the list of data within the groups is non-limiting. The disclosed embodiments may utilize any data within the data repository 115 to analyze patient data and provide the alerts, conclusions, and displayed data disclosed within the disclosed embodiments.

Returning to FIG. 1, the disclosed embodiments may further include one or more software engines and/or software modules which may, as non-limiting examples, include a time-zero calculation software 125 configured to determine time zero, according to the data for each patient stored in the data repository 115, as described in more detail below. As further seen in FIG. 1, the disclosed system may also include a software engine for recovery time zero 140, according to the methods described in more detail herein.

FIG. 1 also demonstrates an alert software 130 that may be configured to transmit and display alerts to providers, as certain criteria are identified, as disclosed in more detail herein. In some embodiments, the alerts software 130 may be configured to generate a text-based alert, which may be transmitted to a provider's personal mobile device or email account. In some embodiments, the provider may access a personal account to provide settings for when and how the criteria or threshold is reached, and the alert should be transmitted. The location of the providers is irrelevant to receiving the alerts; the alert may be available through text, GUI, phone message, etc., which may be set by the user.

In other embodiments, the alerts software 130 may be configured to generate and transmit an alert for display on the near real time display 135 described below. The alert may be triggered according to the algorithms and/or method steps described in more detail below.

As further seen in FIG. 1, the disclosed system may further include near real time display software 135, including logic to constantly and/or intermittently select data from the data repository 115, execute the algorithms and/or method steps described herein, and display the results on one or more client devices, possibly operated by the providers, and coupled to the disclosed system. As with the alerts described above, regardless of the providers' location, they may still have access to the information within the data repository 115 through use of the real time display software 135.

The disclosed system may be constantly updating the data repository 115, by pulling data from the EMR software 105, ADT software 110, or other associated systems for all patients for whom records exist. The near real time display software 135 may then be updated to reflect any new data received. As a non-limiting example, this data may be updated every 15 minutes.

In some embodiments, the software engines and/or modules may analyze the data received in order to implement machine learning algorithms to test the existing data and data logic within the system. As a non-limiting example, the data returned to the data repository 115 may be used as input, determined factors, and parameters to be input into a machine learning algorithm, allowing the system to be self-learning, so that, for example, thresholds determined may be adjusted based on the machine learning, or that false positives and false negatives may be detected to improve both alert performance and clinician confidence.

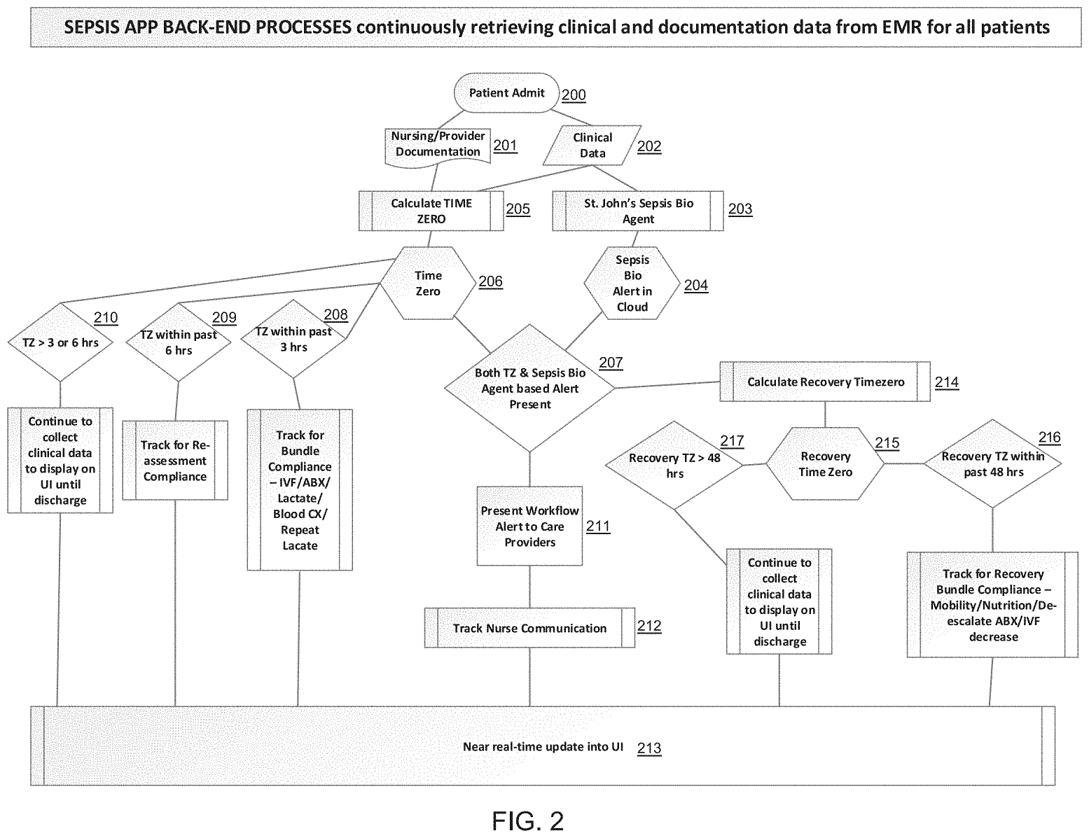

FIGS. 2-4 demonstrate high level flow charts including several algorithms that may be executed for a live sepsis patient. Turning now to FIG. 2, in step 200, patients may be admitted to a hospital, possibly in the context of emergency department or other in-patient scenarios, as non-limiting examples, and may be observed and monitored during their stay.

Providers may interview the patients as they are admitted to gather information, take various vital signs, make general observations, run labs, make diagnoses, etc., and enter this data into the disclosed system 100. As non-limiting examples, the disclosed software and data repository 115 may process the received patient data at the time of admission, and generate data, possibly in the form of data records, for input into the EMR 105 or ADT 110 systems. As described above, to avoid this data being localized at the point of entry, this data may be automatically transmitted from the terminal, to be stored as aggregated data within the data repository 115.

In Steps 201 and 202 of FIG. 2, the aggregated data within the data repository 115 (e.g., nursing/provider documentation 201 and clinical data 202) may then be available to all software within the disclosed system 100. As seen in Step 207 of FIG. 2, this software may work in combination to generate alerts for providers, alerting them to patients for which a sepsis clinical decision support alert 204 and a time zero 206 have been generated and validated.

Before generating the alert in Step 207, the disclosed system may utilize two separate algorithms, which may analyze the aggregated data for each individual patient to first, identify indications of sepsis and set an alert in Step 204, then calculate time zero in Steps 205 and 206. These two algorithms may then work in tandem to generate the alert of the identified sepsis indications and time zero in Step 207. This alert may then be displayed on the near real time display software 135, as seen in Step 213.

As seen in Steps 202-204 of FIG. 2, the first of these two algorithms may utilize a St. John Sepsis Bio Agent algorithm, which may utilize the data generated from the EMR system 105, or any other related system, as stored within the data repository 115, to generate a clinical decision support alert for sepsis. This agent may draw upon the best published evidence and may further use cloud computing with big data analytics to screen high-risk patients and generate a sepsis bio alert, possibly in the cloud, to alert clinicians of potential cases of sepsis, as demonstrated in Step 204.

In some embodiments, the St. John Sepsis Agent may be installed and run in silent mode within a production database used within the disclosed system. Using statistical analysis of the existing data in the data repository 115, the disclosed system may assess frequency and location of sepsis alerts as well as common measures of test performances. The system may then generate a sepsis bio alert within the cloud, as demonstrated in Step 204. In some embodiments, machine learning algorithms may be used to identify features within existing cases of sepsis, and learn from this existing data how to identify indications for new cases of sepsis.

As seen in Steps 201-202, and 205-206, the second algorithm may be configured to calculate time zero from the patient data stored in the data repository 115. In some embodiments, this second algorithm may be built and/or developed to include a predictive model, including generated predictive model values used to reduce the number of false positives and false negatives for detecting indications of sepsis within the first algorithm, and therefore may statistically validate the first algorithm. Thus, in some embodiments, the second algorithm may act as a “catch,” for situations where the first algorithm alert fails to correctly detect indications of sepsis, incorrectly generates an alert of existing sepsis, etc., thereby providing another method of indications of severe sepsis and determining time zero when the first algorithm fails to fire an alert for sepsis indications, or incorrectly fires an alert for detected indications of sepsis.

In some embodiments, the second algorithm may be a standalone algorithm used to identify indications of severe sepsis and time zero, generate alerts, and/or generate the disclosed dashboard, using the near real time display software 135, to display it to providers, as described in more detail below.

Returning to steps 201-202 and 205-206 of FIG. 2, the disclosed system may calculate time zero according to the an earliest reference point based on a latest time stamp of documentation of infection/sepsis, as created when the patient was admitted or using any additional related information in the aggregated data within the data repository 115, which is relevant to the time zero calculation.

Time zero may include the presentation time of signs of severe sepsis and may be calculated for every patient when possible. In some embodiments, the criteria for calculation of time zero may include criteria based on the same criteria that coders use manually to abstract and report time zero to Centers for Medicare and Medicaid Services (CMS).

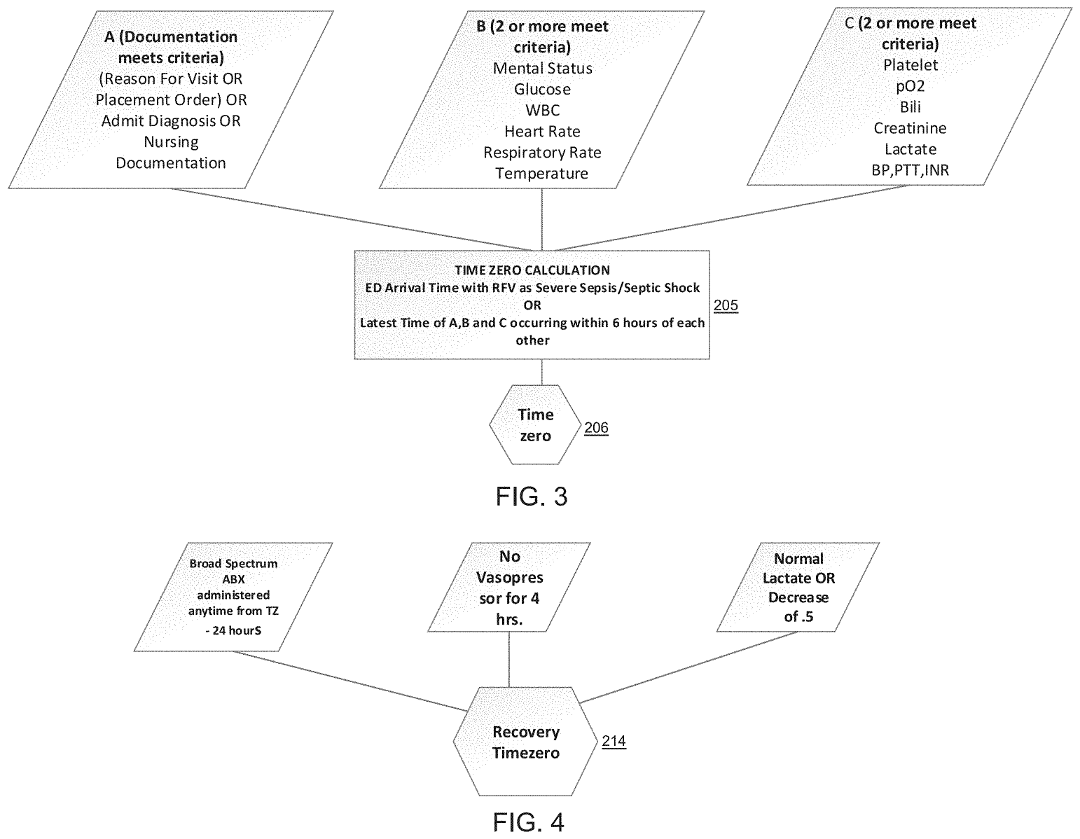

Turning now to FIG. 3, one or more criteria may be used to calculate time zero in steps 205-206. In a first non-limiting example seen in Step 205 of FIG. 3, time zero may be calculated by determining, possibly using the data in the data repository 115, an arrival time of a patient in an emergency department with symptoms of severe sepsis or septic shock as a reason for visit. The disclosed system may then identify time zero as the time of arrival of the patient within the emergency department.

However, as further seen in Step 205 of FIG. 3, a second method of calculating time zero may include identifying a latest time of the concurrence of three separate criteria within 6 hours of each other. These three separate criteria may include a list of elements where documentation meets a specific criteria, where a first list of SIRS elements includes at least two element meeting a specific criteria, and/or where a second list of SIRS elements include at least two elements meeting a specific criteria.

As seen in FIG. 3, the list of elements where documentation meets a specific criteria may include: documentation for the patient's reason for visit or placement order; an admit diagnosis; or nursing documentation related to the patient or patient's condition. In some embodiments, not shown in FIG. 3, the documentation may further include documentation of: the patient's arrival time if the patient was admitted with symptoms of severe sepsis or septic shock, or symptoms of severe sepsis or septic shock; direct admission to the floor as a result of symptoms; a clinician adding a diagnosis to a problem list; or documentation of a suspected source of infection occurring alongside evidence of organ dysfunction or failure (i.e., elevated lactate).

As further seen in FIG. 3, non-limiting examples of a first list of SIRS elements where at least two elements meet a specific criteria may include: a patient's mental status; an abnormal glucose; an abnormal white blood cell (WBC) count; an elevated heart rate; an abnormal respiratory rate, and/or higher or lower than normal temperature (i.e. fever, hypothermia).

As further seen in FIG. 3, non-limiting examples of a second list of SIRS elements where at least two elements meet a specific criteria may include: an abnormal platelet count; an abnormal pO2; an abnormal Bilirubin (e.g., high amount of orange-yellow pigment in blood, indicating jaundice, anemia, liver disease, indicating that red blood cells are breaking down); abnormal creatinine levels; abnormal lactate levels; Abnormal Partial Thromboplastin Time (PTT)/Prothrombin Time (PT, indicating abnormal times for blood to clot), and abnormal INR levels.

Thus, if the disclosed system (e.g., the time zero software 125) determines that the patient has severe sepsis or septic shock, or that additional documentation and other clinical criteria are outside of a normal range, indicating sepsis (e.g., severe sepsis or septic shock), the system may automatically determine that the patient has exhibited symptoms that may indicate sepsis, and may automatically set time zero, thereby improving measures of test performance, bundle compliance, and intensive care unit length of stay.

In some embodiments, the documentation and/or SIRS criteria outside of normal range may be retrieved and analyzed by the disclosed system, possibly the time zero software 125, executing a natural language query to identify, within the data repository 115, certain documentation data and or medical terms from the aggregated data, possibly stored within different medical logs within the system (e.g., EMR and/or ADT records), in order to identify key terms and associated value data associated with the key terms.

For example, a search and analysis of the data within the data repository 115 may indicate, according to current healthcare trends, that certain keywords (e.g., temperature, heartrate, blood pressure, oxygen level, etc.) may give a best indication of sepsis and/or time zero within an admitted and monitored patient. The system may therefore search for any associated keywords, as well as their associated values, and use the results to analyze, within the searched data, data that meets specific criteria to identify patient records that indicate the presentation of severe sepsis and/or time zero, as disclosed above.

Thus, by combining the two algorithms described above, the disclosed system may more accurately identify indications of sepsis and measure and determine the earliest period of time for time zero using the aggregated data within the data repository 115. The first algorithm may generate a first possible alert for sepsis according to the aggregated clinical decision support, and may generate an alert of possible sepsis, and the second algorithm may statistically validate the first algorithm by independently identifying indications of severe sepsis and calculating time zero, as set forth above, thereby providing a predictive model, including generated predictive model values used to reduce the number of false positives and false negatives for detecting sepsis within the first algorithm.

The combination of the two algorithms may further identify the earliest time zero, then confirm the actual time zero. As a non-limiting example, as additional data is aggregated within the data repository 115, the system may continually analyze new data points to determine the earliest time zero, so that if there was a diagnosis or incorrect initial time zero determined, the system will update to reflect the correct time zero, thereby avoiding the loss of time, and allowing providers to take action as needed within a recommended 180 minute window, described below.

Using the automatic identification of indications of sepsis in Step 204, and its associated time zero in Step 206, the disclosed system may continue to improve several key indicators, including superior enhanced timing of the alert to allow for compliance with national standards for the early diagnosis and treatment of sepsis within a 180-minute window, and may improve bundle compliance, and may further reduce ICU length of stay and mortality from sepsis. This 180 minute window has been determined by industry standards (e.g., EMS, CMS) to be an ideal window in which to supply bundle compliance, described below.

The bundle compliance referred to above may refer to sepsis treatment compliance bundles, such as the SEP-1 bundle as a non-limiting example, wherein, if providers have identified indications of sepsis in an admitted patient, they may then begin providing and implementing the necessary steps for treatment for the patient. The disclosed embodiments may include the SEP-1 measure, as a non-limiting example, to accomplish the bundle compliance referred to above. The disclosed system may therefore monitor SEP-1 bundle compliance with essential treatment of sepsis, as established by industry standards (EMS, CMS), in an effort to give sepsis patients a better chance of reducing mortality.

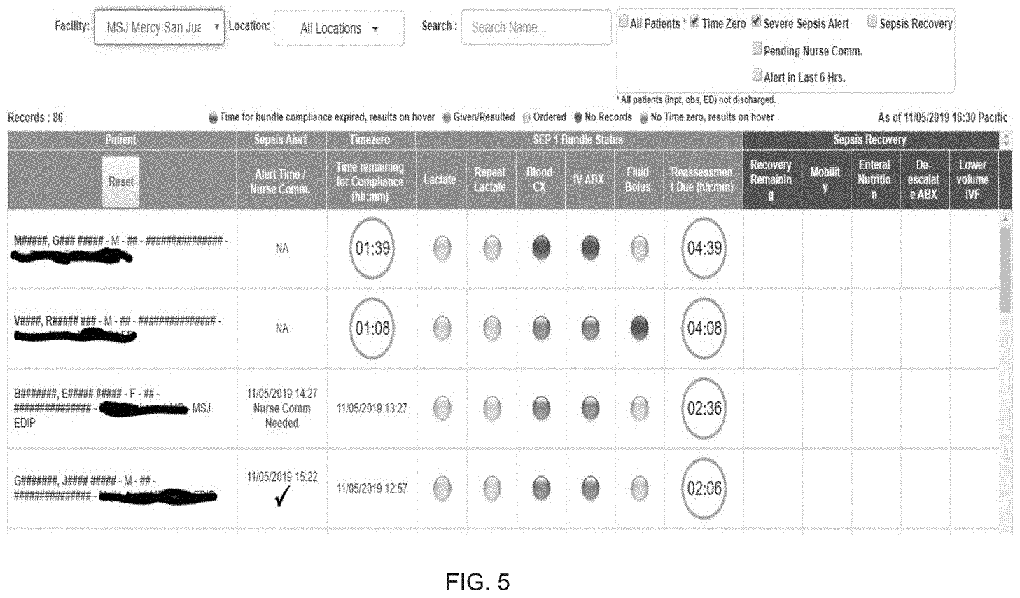

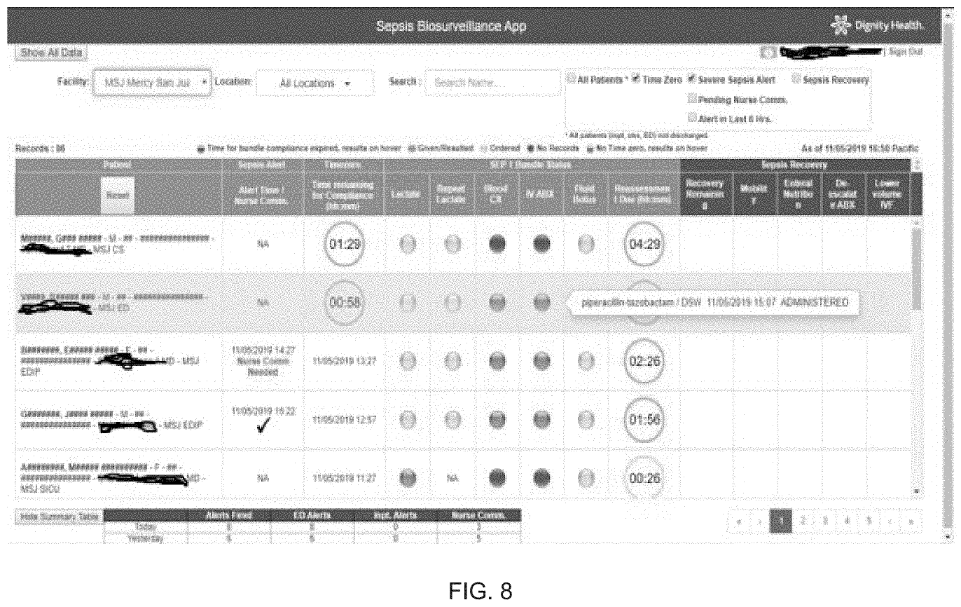

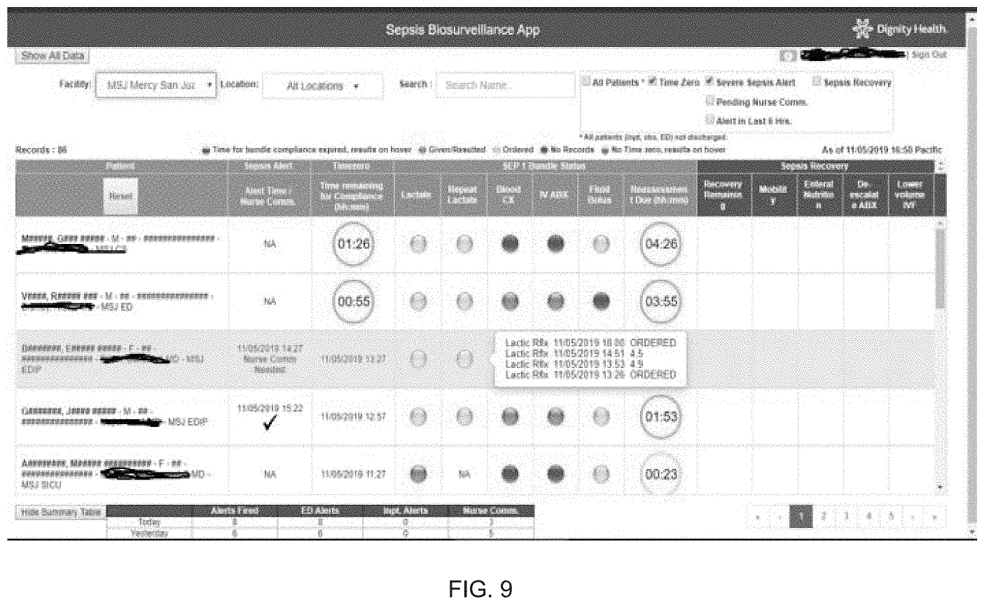

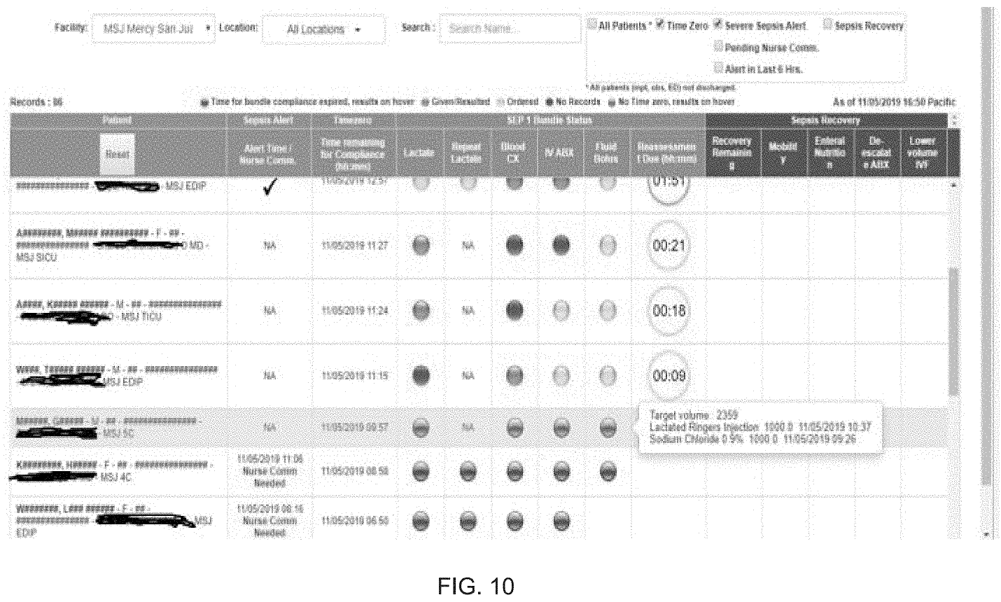

As non-limiting examples seen in FIGS. 5-10, the SEP-1 measure for bundle compliance may include lactate, repeating lactate, blood culture (CX), intravenous antibiotics (IV ABX), and fluid bolus. The implementation of bundles (e.g., SEP-1 bundles), is dependent upon a physician order, which occurs only after a bedside nurse has notified the attending physician of the alert. Thus, such communication, described in more detail below, is essential for bundle compliance. The providers may then continue measuring and monitoring, via the dashboard disclosed in more detail below, the progress of the sepsis bundle patients' identification of time zero.

Returning now to step 208 of FIG. 2, once time zero has been determined for patients which are identified as having sepsis, the disclosed system may determine if the earliest time zero occurred within the past three hours, within the past six hours, or whether the earliest time zero occurred greater than three or six hours previous to the determination of time zero.

If the system determines that the automatically identified time zero occurred within the last 3 hours, the system may automatically track, possibly using the user interface described in detail below, for bundle compliance with the recommended bundle. As seen in Step 208, this tracking may include monitoring the progress of intravenous fluids, antibiotics, lactate, blood cultures, and repeat lactate, as needed.

Ideally, the identification of time zero happens within an hour of time zero. If the automated identification of time zero determines that time zero occurred within the most recent hour, this allows for the timeliest implementation of the sepsis resuscitation bundle described herein.

As seen in Step 209 of FIG. 2, if the automated identification of time zero determines that time zero occurred within the past six hours, the system may automatically track, possibly using the user interface disclosed in detail below, for reassessment compliance.

As seen in Step 210 of FIG. 2 if the automated identification of time zero determines that time zero occurred at a time greater than 3 or 6 hours prior to the identification of time zero, the system may automatically continue to collect clinical data to display the clinical data on the user interface until the patient is discharged.

Returning now to Step 207 of FIG. 2, the disclosed system may be configured to receive the results of the sepsis bio agent and the time zero software 105 to determine if there is a sepsis bio alert in the cloud (Step 204) and whether time zero has been calculated (Steps 205 and 206). As seen in Step 207, if an alert is present based on the sepsis bio agent alert, and if the time zero software 125 has identified a time zero, the disclosed system may use the resulting data and the statistical analysis of resulting alerts, to automatically analyze the received data and set one or more software triggers for presenting at least one workflow alert to be transmitted to care providers.

Put another way, in some embodiments, the disclosed system may be configured to regularly test these parameters to “listen for” or otherwise determine if any or a combination of the criteria set forth above are out of range. If so, the disclosed system may be configured to generate and transmit an alert to any combination of providers that may have been pre-designated for receiving the alert.

Turning now to Step 211 of FIG. 2, in response to the system generating an alert in Step 207, the disclosed embodiments may be configured to present a workflow alert to care providers, and in step 212, may begin tracking nurse communications. More generally, once a predictive alert is fired within the system, the system may begin monitoring the treatment segment of the disclosed system and any associated communications.

As seen in FIG. 2, to accomplish this, the disclosed embodiments may transmit the data received in the method steps above for display on the near real time display 135. In step 213, the disclosed system may then update a user interface on the near real time display 135 to include any updated data (e.g., data received and processed within the last 15 minutes).

Turning now to FIGS. 5-12, the current embodiments include a near real time display software 135 within the application, which compiles the aggregated data within the data repository 115, and displays the results as information about a patient or a patient's condition on a user interface in real time or near real time (e.g., every 10-15 minutes). In some embodiments, the data may be displayed on a dedicated monitor in a common area in the emergency department, or ICU.

As further seen in FIGS. 5-12, this user interface may display a table, in which the columns are divided into information about the patient, the sepsis alert, the determined time zero, the SEP-1 bundle status, and the sepsis recovery of each patient. Each of these sections may be subdivided into additional columns, as described in detail below, which provide specific information about a patient, their diagnosis, their current treatment, and their recovery, thereby providing providers (e.g., doctors and nurses) with a quick glance at the inner workings of the system to determine the meaning and significance of each column, including various elements that have contributed to the sepsis, what treatments may be used to help the patients overcome the sepsis and speed recovery, etc.

As demonstrated in FIGS. 5-12, the UI may display a list of patients (e.g., those patients diagnosed with sepsis), for those patients that have not been discharged, as well as patient data including name, faculty, location, etc. as well as observations by the providers and current treatment for the displayed patients. In some embodiments, this additional data may be available to the providers after hovering a cursor over the appropriate column for the patient data, as described in more detail below.

As seen in FIG. 5-12, the user interface may include one or more filters, used to display a list of patients according to facility or location, or to search for a specific patient using a search field. The user may also use that same search field to find a list of patients for a specific attending physician, etc. As further seen in FIGS. 5-12, additional filters may allow users to display all patients, patients according to time zero, patients with a severe sepsis alert, patients in sepsis recovery (described below), patients who have pending nurse communications, or patients who have had an alert trigger in the last 6 hours. The results of the display and filtering may be further sorted by hovering over a column header to reveal a sort icon. The user may then click the sort icon to further sort the search results.

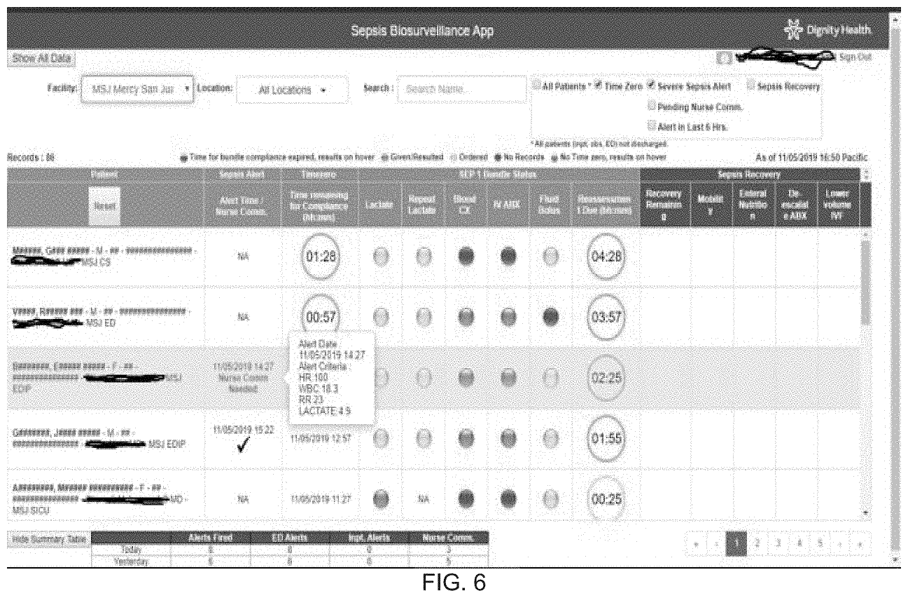

The dashboard may display information for the sepsis alert, and may be configured to display information about a patient and the patient's condition relative to the sepsis, as available in routine EMR workflow, in real time. As seen in FIGS. 5-12, the dashboard may further include the sepsis alert time in a cell associated with the patient record under the sepsis alert column. This may include data within a cell associated with the patient and displayed in the Sepsis Alert/Alert Time/Nurse Comm. column, and may include the most current sepsis alert date/time. If there is no sepsis alert associated with the patient, and no sepsis alert communications are expected, ‘NA’ may be displayed in the Sepsis Alert column. FIG. 6 demonstrates a feature of the disclosed user interface allowing users to hover over the date/time stamp for each alert, which provides additional data about the alert, including alert time, factors that contributed to the alert, etc.

Because the nursing staff owns the primary workflow and responsibility to notify the provider of a severe sepsis alert, the disclosed system measures and monitors nurse communication within the disclosed system. The results of this monitoring and communication are also displayed on the sepsis dashboard user interface. Thus, as seen in FIGS. 5-10, the user interface may be configured to further display details about nurse communications, associated with the identified sepsis and alerts, to physicians. By doing so, the timeliness of physicians reaching bundle compliance may be improved, and efficiency increased.

FIGS. 5-12, demonstrate indicators of such communication associated with the alert time has occurred (e.g., a checkmark when communication documentation has been completed), or an indicator that nursing communication has not yet occurred. This column may emphasize the importance of knowing when the alert time was first detected, and when the details were communicated to the doctor by the nurse. In some embodiments, nurse communication details may appear in an individual column/cell.

FIGS. 5-12 further demonstrate a time zero column, providing a time zero for each patient record. As non-limiting examples, this column may include a display of time zero, and/or a time remaining for compliance, displayed as hours and minutes, in FIGS. 5-10. In light of the industry standards discussed above, it is important for the provider to take some action within 180 minutes (3 hours) from the early detection of sepsis. This column may therefore motivate providers to take action and remain within bundle compliance. This column may display, for each patient record, the time zero determined by the time zero software 125, and may display time zero, possibly as a closing circle or as a progress bar.

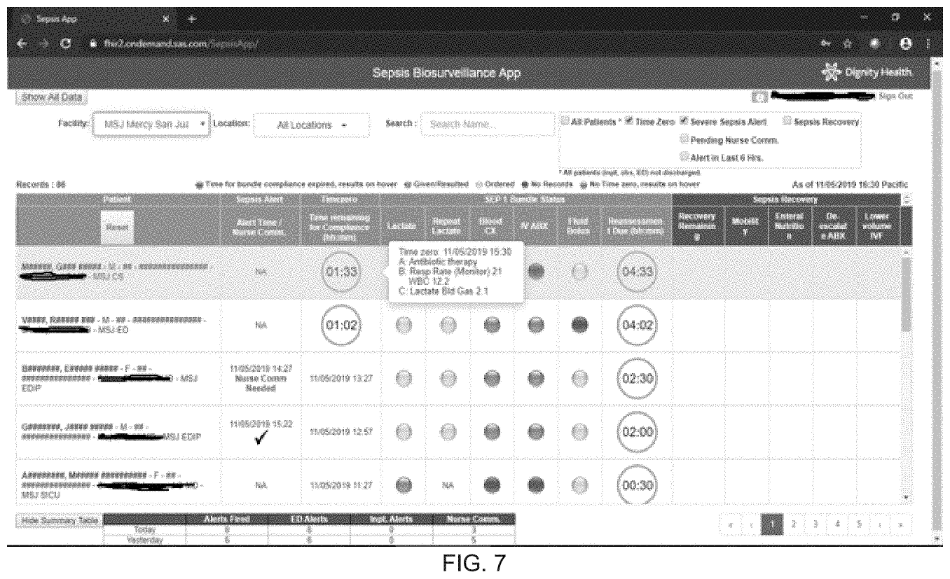

The time remaining for compliance displayed in the user interface may also provide notice of the time of sepsis alert, and the time remaining for bundle compliance. In some embodiments, patients associated with a sepsis alert or a calculated time zero are displayed by default and the results are sorted descending by ‘Time Remaining for Compliance.” As seen in FIG. 7, the user may hover over the data within the time zero column to view additional details, including when time zero occurred, and associated data used to determine the existence of sepsis and the factors used in calculating time zero.

The system may then use the data from the data repository 115, as gathered from the EMR 105 and ADT 110 systems, to create a compliance bundle template for each patient, and populate each patient record with the regularly updated data. To better communicate recommended actions, the disclosed embodiments may use the user interface to capture a snapshot of status of the SEP-1 bundle. Thus, as seen in FIGS. 5-12, the disclosed system may generate a column/cell for each of the steps of SEP-1 compliance, providing data about the current status of each of these recommended steps.

It should be noted that the disclosed dashboard does not instruct physicians on the treatment of the patient, but instead only displays the recommended steps according to the SEP-1 bundle. However, following particular compliance as directed by this tool results in improved protection of sepsis patients. In some instances, physicians may determine, based on their diagnosis and analysis of the each individual patient, that some steps of the SEP-1 measure are not necessary. As a precaution, the user interface provides a notification to doctors that certain elements of the SEP-1 bundle have not been addressed, but the physician ultimately makes the decision of whether to move forward with those steps in the time remaining for compliance since time zero. The providers may then provide treatment and continue to monitor the progress of each patient. As data is input into the system, the dashboard may be updated in real time.

In some embodiments, the status of various SEP-1 bundle steps are indicated by a colored dot or bubble. By reviewing the snapshot of the status of each of these steps, the physician may achieve compliance. As a non-limiting example, in the disclosed embodiments, may be presented in red, yellow or green for 3-6 hours after time zero. At the end of that active bundle's completion time’, the bubbles may change to blue. It should be noted, however, that the indication of SEP-1 status may be indicated by any means known in the art for status indicators within a graphical user interface.

In some embodiments, no bubble will be displayed for some of the SEP-1 bundle status items. This indicates that there is no record for that bundle item, and/or that the bundle item was not addressed. For example, the doctor may have decided not to act on that item. If the status for a particular cell is empty at the expiration of time for compliance, the cell for that item will remain empty.

In some embodiments, the bubbles may be colored gray, indicating that no time zero was determined for that bundle item. However, orders or results for these bundle items may still exist, and the details for such orders or results may be available by hovering a cursor over the appropriate items.

In some embodiments, the bubble for a bundle item may be red. A red bubble may indicate that the data repository 115 has received no records, audits etc. associated with the patient record for that bundle item, and that perhaps an assessment or reassessment is required for the associated patient. A red bubble only indicates that the bundle item has not been completed, and is not necessarily an alert. Thus, a physician may still provide the patient with the particular bundle item in the time remaining displayed on the GUI.

A yellow bubble in the disclosed embodiments may indicate that an order has been given for a particular bundle item, and that the bundle item for which the order has been placed is currently being processed.

A green bubble in the disclosed embodiments may indicate that the particular bundle item has been administered and/or that the results of the particular bundle have been received.

A blue bubble in the disclosed embodiments may indicate that a time for bundle compliance has expired. As a result, the bubbles may appear red, yellow, or green while there is still time remaining for compliance, but once that time has expired, all existing bubbles may turn blue.

As seen in FIGS. 6-10, the disclosed dashboard may further include a remaining time available for reassessment. As noted above, reassessment may be available for an additional 180 minutes past the initial 180 minutes established from the identification of time zero. The reassessment data may include data for reassessing the status of the patient each hour after the completion of the initial 180 minutes. Thus, unlike the time zero time remaining for compliance (3 hours), the time for reassessment may be 360 minutes, or 6 hours, within which the provider may reassess the SEP-1 measure. Like time zero, this time remaining for reassessment may display notice of the time of sepsis alert, and the time remaining for reassessment within the sepsis bundle (e.g. SEP-1 ) compliance.

An additional time measurement displayed on the GUI may include a start time for reassessment. In light of the research discussed above, it is important for the provider to take some action within 360 minutes (6 hours) from the early detection of sepsis. Similar to the time remaining GUI element, the reassessment GUI element may include a circle or progress bar, indicating the time remaining for reassessment, counting down the time remaining for bundle compliance, namely 3 hours from time zero for the bundle, 6 hours from time zero for reassessment.

As seen in many of the example embodiments seen in FIGS. 5-12, the dashboard may be configured to present a snapshot, in real time, of the current status of sepsis alerts, time zero, time remaining, nurse communications, and the status of each of the SEP-1 bundle items. However, in the disclosed embodiments, the details for any of these items may be accessed by the provider by hovering over any of the items.

As non-limiting examples, the user may hover over: the alert time to determine the time and reason for the alert; the nurse communication needed or completed item to view access to the documentation provided by the nurse; the time remaining for compliance or reassessment due to view details of the time and reasons for time zero or reassessment, and the like.

Likewise, for the SEP-1 bundle status items, the orders and results for each element will be displayed in the hover. A user may hover over any bubble to see the details for any selected item (e.g., the orders and results associated with that item). This list will continue to update in real time through the patient's stay, and in some embodiments, may display the 4 most recent orders or results. The user may continue to hover over blue bubbles to view continuing results.

These examples are non-limiting. In some embodiments, any item within the displayed user interface may be hovered over to receive current or most recent information in the data repository 115 related to the item over which the user hovers.

The providers may continue to provide and monitor the treatment, within the 3 or 6 hours since the onset of sepsis. Returning now to Steps 214-215 of FIG. 2, the providers may then determine that the patient has entered a recovery phase. The recovery phase may be an indication of stability after treatment for severe sepsis and a prompt to consider advancing the orders for the patient toward transfer or discharge. However, it should be noted that even if the timer for compliance has expired, and that the person is no longer being tracked (i.e., the patient is out of compliance), this does not mean that the person is in the recovery phase and that they can start recovery. They may still be in some kind of sepsis, and require further treatment.

In disclosed embodiments, the recovery phase may be determined using an algorithm, as seen in FIG. 4. Prior to the start of this algorithm, any combination of the system and the providers may determine that the patient has had a severe sepsis alert, and the system may automatically calculate and display time zero, as disclosed herein. As the patient progresses with their treatment of the sepsis, the system may execute the algorithm shown FIG. 4. This algorithm constantly monitors those sepsis patients to see whether they have achieved recovery, and if so, calculates a recovery time zero, a point of time that they are deemed to be recovering. The system may then start a recovery countdown from the point in time that recovery time zero is identified. The system may determine recovery time zero according to the following determinations.

A first step in this algorithm, as seen in FIG. 4, may include the providers determining, or the system automatically determining, that broad spectrum antibiotics have been administered any time from time zero to 24 hours. If so, recovery time zero may be started in step 214.

Another step or factor in determining whether the patient is in the recovery phase, and to begin recovery time zero, is to determine whether there have been vasopressors for 4 hours, as demonstrated in FIG. 4. If not, recovery time zero may be started in Step 214.

Another step or factor in determining whether the patient is in the recovery phase, and to begin recovery time zero, is to determine whether there has been normal lactate or decrease of 0.5, as demonstrated in FIG. 4. If so, recovery time zero may be started in Step 214.

Returning to FIG. 2, in Step 216, if the calculated time zero is within the past 48 hours, the system and/or the providers may track for recovery bundle compliance, including mobility, nutrition, de-escalation of antibiotics and decrease of intravenous fluids, as described in more detail below. However, in Step 217, if the recovery time zero is greater than 48 hours, the system and/or providers may continue to collect clinical data to display on the user interface until the patient is discharged.

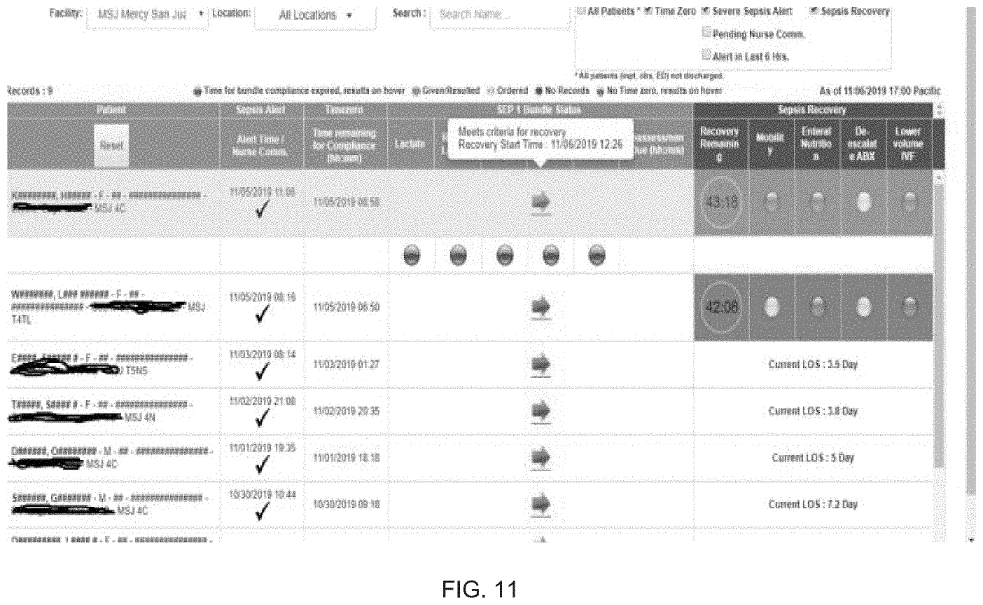

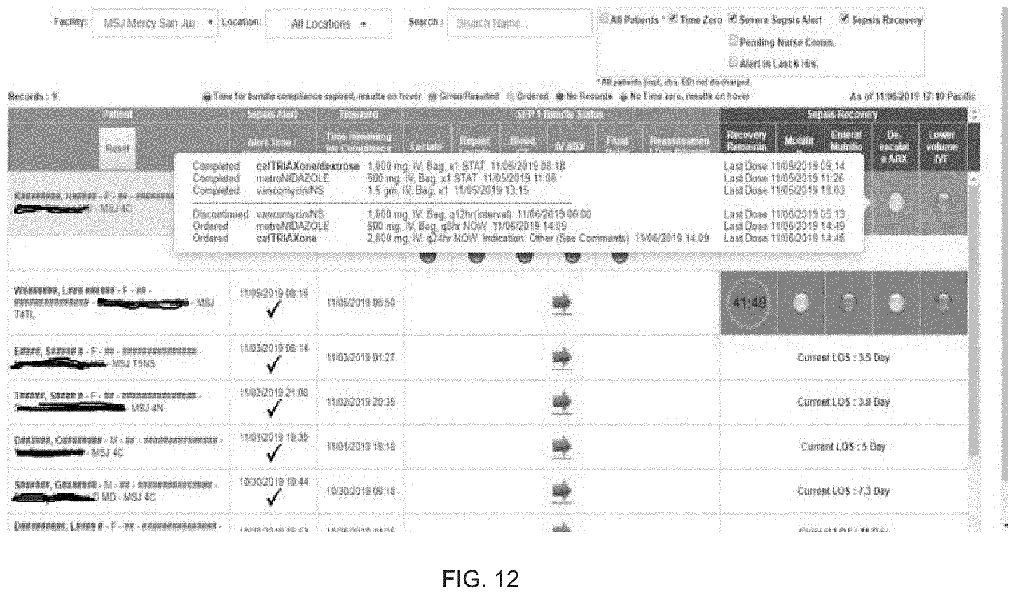

Once recovery time zero has been identified, the system may automatically update the user interface for the patient record to reflect that the patient has entered the recovery phase, and that recovery time zero has begun, as demonstrated in FIGS. 11-12. However, unlike time zero and alerts, there is no interruptive notification for recovery status. As above, in some embodiments, a checkbox filter may be used to sort and filter the patients eligible for recovery, so that these recovery patients are displayed by default.

In some embodiments, when the user enters the recovery phase, the SEP-1 bundle elements may be hidden on the user interface as soon as the patient meets the recovery criteria, and may display a green arrow icon, as seen in FIGS. 11-12. In some embodiments, a user may click the green arrow icon to display resuscitation details. In the example embodiments shown in FIGS. 11-12, clicking on the green arrow icon may display the associated blue bubbles for SEP-1 compliance, and the user may hover over these bubbles to receive the details and review the treatment of sepsis during this phase.

The user interface demonstrated by the non-limiting examples in FIGS. 11-12, include an update to include the calculated recovery time zero once the patient is achieving recovery and has entered a recovery phase. The user interface may be further updated to include recovery compliance and reflect the recovery time remaining, based on the calculated recovery time zero.

As seen in FIGS. 11-12, in some embodiments, the recovery phase may be indicated within the sepsis recovery portion of the dashboard by shading the entire portion of the sepsis recovery portion. In some embodiments, this shading may be in green. This green shading may continue for the 48 hours during which the patient is in the recovery phase, a 48 hour window during which length of stay, possibly within an ICU unit, may be reduced.

The disclosed system may therefore include a recovery bundle, which may be similar to the sepsis bundle, but instead of managing the sepsis symptoms and getting the patient out of sepsis, the goal of the recovery bundle is to start recovery and to move the patient towards discharge or transfer from the unit or the hospital. This sepsis recovery bundle may include orders that can impact the patient's length of stay.

Once recovery is started, the recovery compliance elements may allow the providers to track, and may include, as non-limiting examples: Mobility, Enteral Nutrition, De-escalation of antibiotics, and lower volume intravenous fluids. Recovery compliance for mobility may be achieved when the patient achieves level 3 for mobility or higher, or is ambulating. Recovery compliance for enteral nutrition may be achieved when at least a clear liquid diet is ordered. Such a clear diet order indicates that the patient has come out of sepsis. Tube feeding may be considered a clear liquid diet, according to standard recovery compliance principles. Recovery compliance for de-escalation of antibiotics may be achieved when the patient has no antibiotics, or fewer antibiotics ordered than during the acute sepsis treatment. Recovery compliance for lower volume of intravenous fluids may be achieved when the patient has had less than 960 mL of maintenance intravenous fluids in the last 24 hrs (TKO rate).

Thus, as seen in FIGS. 11-12, in addition to a countdown for the given timeframe (48 hours from the identified recovery time zero) the updated user interface for the patient record may include a countdown for the multiple elements for the recovery bundle.

As seen in FIGS. 11-12, the updated user interface may include recovery compliance elements from a recovery compliance bundle within recovery compliance. Thus, once a patient has entered the recovery phase, the dashboard may be updated. As above, dots or bubbles may indicate the patient's status in the sepsis recovery portion of the dashboard. These bubbles may have yellow or green status, as described above.

For example, for mobility the user interface may display a green bubble when the patient achieves level 3 or higher, or is ambulating. For Enteral Nutrition, the user interface may display a green bubble when a clear liquid diet is ordered. For De-escalation of antibiotics, the user interface may display a green bubble when the patient has no or fewer antibiotics ordered than during acute sepsis treatment, and for lower volume intravenous fluids, the user interface may display a green bubble when the patient has had less than 960 mL of maintenance intravenous fluids in the last 24 hrs. As above, and as demonstrated in FIG. 12, providers may again hover over elements of the display in order to view details for the dashboard.

It should be noted that the algorithms above are applied in the context of the disclosed embodiments for a sepsis patient. However, the algorithm for recovery may also be applied for patients in general, including any or all patients in the hospital.

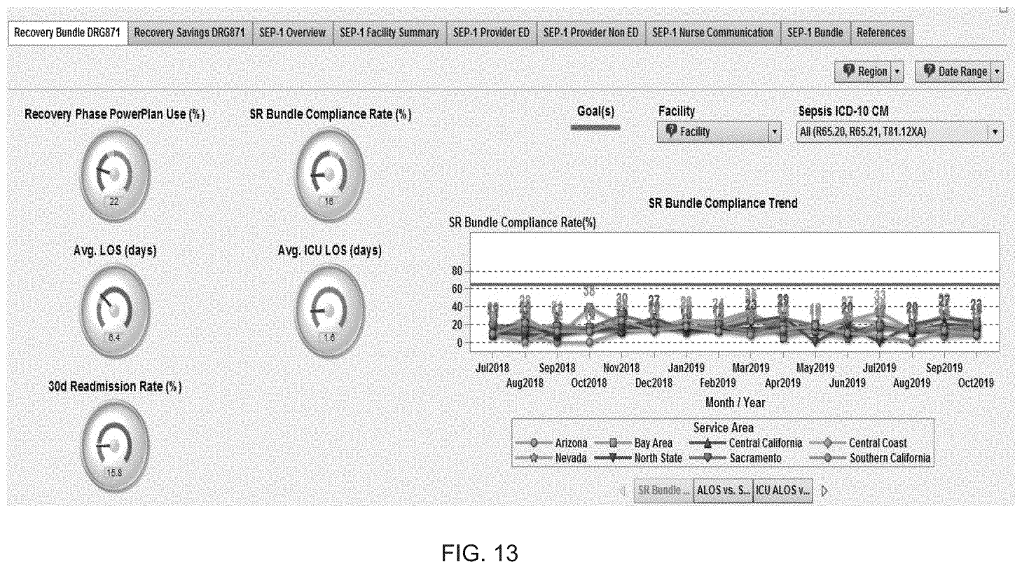

As seen in FIGS. 13-16, the system may further include multiple retrospective dashboards, configured to provide a retrospective analysis of data collected during the normal course of patient care, and within the disclosed system. The system may compile the data disclosed above, and may generate one or more retrospective dashboards to show retrospective trends in different areas.

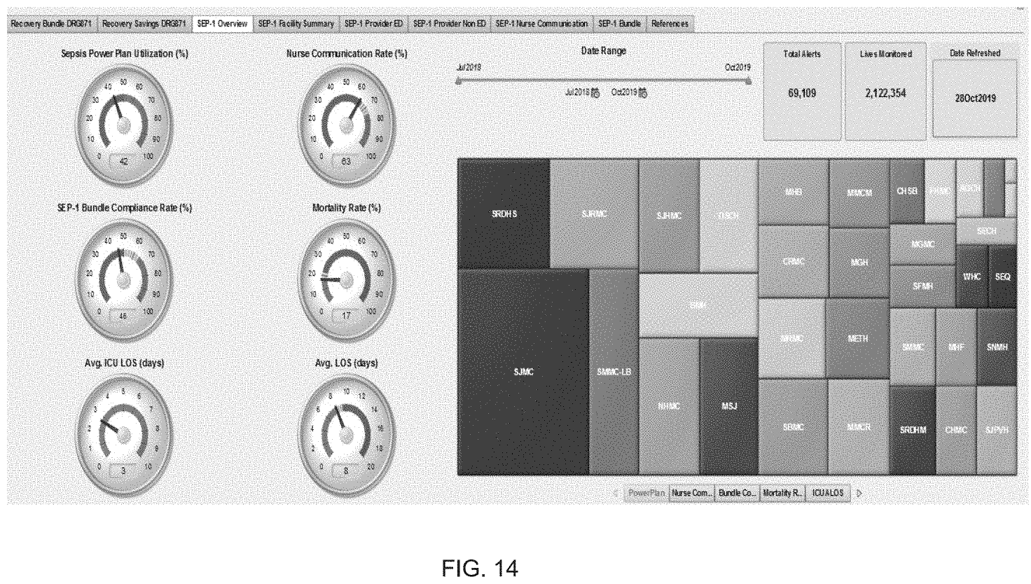

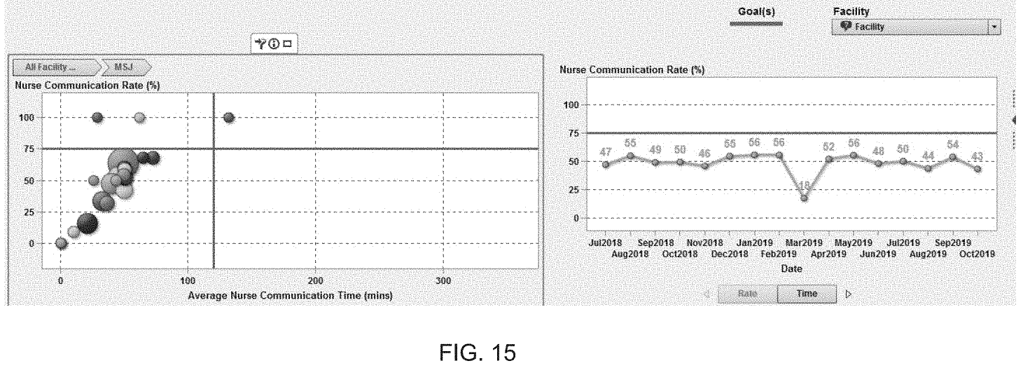

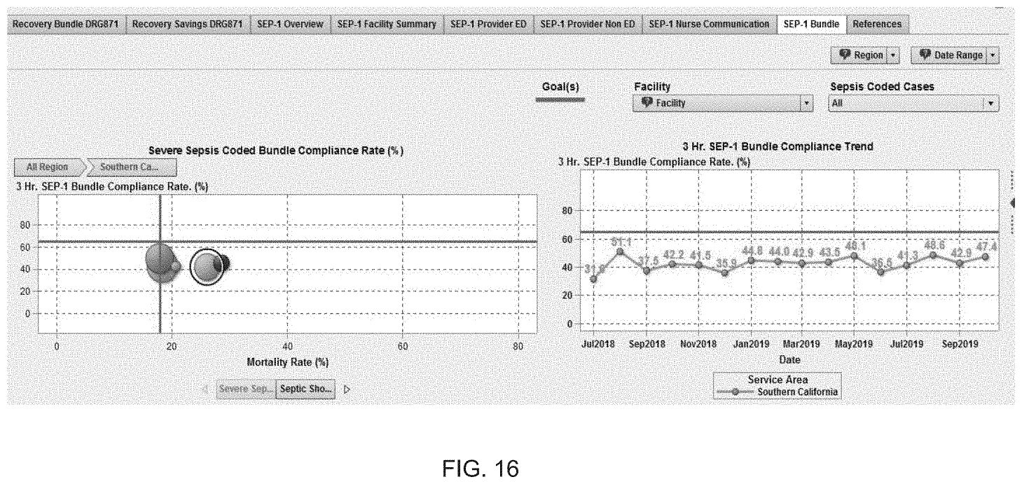

As non-limiting examples, FIG. 13 includes trends for various facilities relating to their compliance with the sepsis recovery bundle. FIG. 14 displays an overview of compliance, length of stay and ICU length of stay, nurse communication, and mortality rate for facilities over a particular date range. FIG. 15 shows trends of average nurse communication time and rate for selected facilities, and FIG. 16 shows trends for severe sepsis bundle compliance for selected facilities.

As seen on these example retrospective dashboards, the system may capture all the data regarding alerts, compliance, nurse communication, and the like to track how providers react, thereby showing a correlation to item mortality or reduced length of stay. The system may cycle and apply advanced analytics from detection, to monitoring, to collecting the retrospective knowledge, and may provide and display feedback so that specific facilities, or all facilities, know how to change their approach.

Thus, the dashboards may provide a quick overview of how a particular clinic, or a particular area within the hospital is doing. This data may provide insights allowing the programs to fine tune and find the best practices, as well as determine how all hospitals within a network are performing.

The present invention has been described in terms of one or more preferred embodiments, and it should be appreciated that many equivalents, alternatives, variations, and modifications, aside from those expressly stated, are possible and within the scope of the invention.

1. A system comprising:
a data repository coupled to a computer network and storing a plurality of data aggregated in association with each of a plurality of patients, the plurality of data comprising:
a documentation data input by at least one healthcare professional; and
a clinical data associated with the patient;
a server, comprising at least one server hardware computing device coupled to the computer network and comprising at least one processor executing instructions within a memory which, when executed, cause the system to:
automatically generate, based on the plurality of data, a sepsis alert identifying an indication of sepsis for a patient in the plurality of patients;
automatically calculate, based on the plurality of data, a time zero for the patient;
automatically monitor a plurality of communications between a plurality of healthcare providers;
generate a Graphical User Interface (GUI) comprising:
an alert of the indication of sepsis and the time zero for the patient;
an indication of the status of the plurality of communications;
a first countdown indicating a first remaining time from the time zero to complete a plurality of sepsis bundle compliance steps for the patient;
a first status indicator for each of the plurality of bundle compliance steps;
a second countdown indicating a second remaining time from the time zero to complete at least one reassessment compliance step; and
a second status indicator for each of a plurality of sepsis recovery compliance steps for a sepsis recovery of the patient.

2. The system of claim 1, wherein the GUI is configured to display:
the first countdown according to a 180 minute countdown from the time zero, and formatted as a number of remaining hours and minutes; and
a GUI element representing the amount of time remaining in the first countdown.

3. The system of claim 1, wherein the GUI is configured to display the first status indicator for each of the plurality of sepsis bundle compliance steps according to:
a first indicator, indicating that a bundle step in the plurality of sepsis bundle compliance steps has been completed;
a second indicator, indicating that the bundle step in the plurality of sepsis bundle compliance steps has been ordered;
a third indicator, indicating that the bundle step in the plurality of sepsis bundle compliance steps is not associated with any records;

4. The system of claim 3, wherein the GUI is configured to display:
the first indicator as a first color;
the second indicator as a second color; and
the third indicator as a third color.

5. The system of claim 1, wherein the GUI is configured to display the second countdown according to a 360 minute countdown from the time zero.

6. The system of claim 1, wherein the second status indicator further comprises a third countdown indicating a third remaining time from a recovery time zero to complete at least one recovery compliance step.

7. A method comprising:
storing, by a server comprising at least one server hardware computing device coupled to the computer network and comprising at least one processor executing instructions within a memory, within a data repository coupled to the computer network, a plurality of data aggregated in association with each of a plurality of patients;
automatically generating, by the server, based on the plurality of data, a sepsis alert identifying an indication of sepsis for a patient in the plurality of patients;
automatically calculating, by the server, based on the plurality of data, a time zero for the patient;
automatically monitoring, by the server, a plurality of communications between a plurality of healthcare providers;
generating, by the server, a Graphical User Interface (GUI) comprising:
an alert of the indication of sepsis and the time zero for the patient;
an indication of the status of the plurality of communications;
a first countdown indicating a first remaining time from the time zero to complete a plurality of sepsis bundle compliance steps for the patient;
a first status indicator for each of the plurality of bundle compliance steps;
a second countdown indicating a second remaining time from the time zero to complete at least one reassessment compliance step; and
a second status indicator for each of a plurality of sepsis recovery compliance steps for a sepsis recovery of the patient.

8. The method of claim 7, further comprising the step of generating, by the server, a predictive model configured to reduce a total number of false positives and false negatives within the system when generating the sepsis alert.

9. The method of claim 7, further comprising the step of automatically identifying the indication of sepsis according to:
an arrival time in an emergency department of the patient with severe sepsis or septic shock; or
a plurality of criteria combining:
at least one documentation identifying a reason for visit for the patient, or an admit diagnosis; or
at least two elements meeting a specific criteria.

10. The method of claim 9, further comprising the step of:
determining that the at least two elements are outside of a predefined range; and
displaying, on the GUI, the alert of the indication of sepsis in response to the at least two elements being out of range.

11. The method of claim 7, further comprising the steps of:
responsive to the time zero having occurred within 180 minutes, displaying, by the server on the GUI, a first plurality of GUI elements measuring and monitoring the plurality of sepsis bundle compliance steps;
responsive to the time zero having occurred greater than 180 minutes but less than 360 minutes, displaying, by the server on the GUI, a second plurality of GUI elements measuring compliance with a reassessment; and
responsive to the time zero having occurred greater than 360 minutes, aggregating, by the server, an additional plurality of data for storage in the data repository.

12. The method of claim 7, further comprising the step of:
identifying, by the server, a recovery time zero;
generating, by the server according to the recovery time zero identified, the second countdown for display within the second status indicator on the GUI; and
transmitting, by the server, the second countdown to a client device for display on the GUI.

13. The method of claim 12, further comprising the step of identifying the recovery time zero according to a determination that:
at least one broad spectrum antibiotic has been administered to the patient from time zero to a time 24 hours later than time zero;
no vasopressors have been detected for the patient in the previous 4 hours; or
the patient has demonstrated a normal lactate level, or a decrease in lactate level of at least 0.5.

14. The method of claim 7, further comprising the steps of displaying, on the GUI:
a first recovery bundle compliance step indicating a mobility status for the patient;
a second recovery bundle compliance step indicating a nutrition status for the patient;
a third recovery bundle compliance step indicating a de escalation of antibiotics status for the patient;
a fourth recovery bundle compliance step indicating a level of intravenous fluids status for the patient;

15. A system comprising a server, comprising at least one server hardware computing device coupled to the computer network and comprising at least one processor executing instructions within a memory which, when executed, cause the system to:
store, within a data repository coupled to the computer network, a plurality of data aggregated in association with each of a plurality of patients;
automatically generate, based on the plurality of data, a sepsis alert identifying an indication of sepsis for a patient in the plurality of patients;
automatically calculate, based on the plurality of data, a time zero for the patient;
automatically monitor a plurality of communications between a plurality of healthcare providers;
generating, by the server, a Graphical User Interface (GUI) comprising:
an alert of the indication of sepsis and the time zero for the patient;
an indication of the status of the plurality of communications;
a first countdown indicating a first remaining time from the time zero to complete a plurality of sepsis bundle compliance steps for the patient;
a first status indicator for each of the plurality of bundle compliance steps;
a second countdown indicating a second remaining time from the time zero to complete at least one reassessment compliance step; and
a second status indicator for each of a plurality of sepsis recovery compliance steps for a sepsis recovery of the patient.

16. The system of claim 15, wherein the server is further configured to display, on the GUI the first status indicator for each of the plurality of sepsis bundle compliance steps according to:
a first indicator, indicating that a bundle step in the plurality of sepsis bundle compliance steps has been completed;
a second indicator, indicating that the bundle step in the plurality of sepsis bundle compliance steps has been ordered;
a third indicator, indicating that the bundle step in the plurality of sepsis bundle compliance steps is not associated with any records;

17. The system of claim 16, wherein the server is further configured to display, on the GUI:
the first indicator as a first color;
the second indicator as a second color; and
the third indicator as a third color.

18. The system of claim 15, wherein the server is further configured to display, on the GUI, the second countdown according to a 360 minute countdown from the time zero.

19. The system of claim 15, wherein the second status indicator further comprises a third countdown indicating a third remaining time from a recovery time zero to complete at least one recovery compliance step.
